(19) United States
(12) Patent Application Publication
(10) Pub. No.: US 2023/0015351 A1
(43) Pub. Date: Jan. 19, 2023

(54) SANITIZING AND ODOR ELIMINATION COMPOSITION AND METHOD OF MAKING THE SAME

(71) Applicant: Daniel Aponte, Davie, FL (US)

(72) Inventor: Daniel Aponte, Davie, FL (US)

(21) Appl. No.: 17/375,349

(22) Filed: Jul. 14, 2021

(51) Int. Cl. A61L 9/012; A61L 9/14; A61L 9/04

(52) U.S. Cl. CPC A61L 9/012; A61L 9/14; A61L 9/044

(57) ABSTRACT

The present disclosure relates to a composition for sanitizing and de-odorizing air and method of making the same. The composition may reduce and eliminate airborne bacteria and odor in an environment. The composition may include ethyl alcohol, propylene glycol, an essence such as linalool or linalyl acetate.

### TECHNICAL FIELD

The embodiments generally relate to the field of compositions of sanitizers and deodorizers and methods of sanitizing and deodorizing.

### BACKGROUND

The present disclosure relates to a composition for sanitizing and de-odorizing air or environments and methods of making the same. Sanitizing alcohols in combination with essences may provide a means of sanitizing and de-odorizing air or environments.

### SUMMARY

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The present disclosure relates to a composition for sanitizing and de-odorizing air and method of making the same. The composition reduces airborne bacteria and eliminates odor in an environment. The composition may include an essence as a component.

The present disclosure relates to a composition for sanitizing and de-odorizing comprising 82.9 wt. % ethyl alcohol; 9.8 wt. % propylene glycol; and 7.3 wt. % essence.

The present disclosure relates to a composition for sanitizing and de-odorizing comprising 35-45 wt. % ethyl alcohol; 4-9 wt. % 1,2-propanediol; an essence comprising at least one of linalool or linalyl acetate; propane; and butane.

The present disclosure relates to a method for sanitizing and de-odorizing, the method including the steps of providing an aerosolized admixture of 35-45 wt. % ethyl alcohol, 4-9 wt. % 1,2-propanediol, and essence; and applying the aerosolized mixture to an environment in an effective amount to treat the environment such that airborne bacteria and odor are reduced or eliminated.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. Any accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

The features and advantages of the present disclosure will be more fully described in, or rendered obvious by the following detailed description of the preferred embodiments, which are to be considered together with the accompanying drawings wherein like numbers refer to like parts and further, wherein:

FIG. 1 is a graph shows the effectiveness of different compositions over time.

### DETAILED DESCRIPTION

Reference will now be made in detail to the present embodiment(s), and examples of which is/are illustrated in any accompanying drawings. Whenever possible, the same reference numerals will be used throughout the drawings to refer to the same or like parts. Any specific details of the embodiments are used for demonstration purposes only, and no unnecessary limitations or inferences are to be understood therefrom.

As used in this disclosure, the term “essence” may refer generally to a substance in concentrated form derived from a plant, material, or the like. An essence may include linalool that may provide an appealing scent or aroma and may function as a deodorant. An essence may include linalyl acetate that may provide an appealing scent or aroma and may function as a deodorant. An essence may also include an essential oil, natural oil, perfume, or scent in an optional solution such as alcohol.

As used in this disclosure, the terms “aerosol” or “aerosolized” may refer generally to a substance being suspended in air such that it is considered fine spray. A means of aerosolizing a substance may include converting the substance into a fine spray by providing a substance of small enough particle size that the substance may be moved in a stream of gas or air such that the substance can be dispersed.

The present disclosure provides a composition which may comprise, in a mixture, approximately 82.9 wt. % ethyl alcohol, 9.8 wt. % propylene glycol, and 7.3 wt. % essence such as linalool, linalyl acetate, or as combination thereof. The ethyl alcohol, propylene glycol, and essence may be combined in an admixture.

The present disclosure provides a composition which may comprise, in a mixture, approximately 41.45 wt. % ethyl alcohol, 4.9 wt. % propylene glycol, and 3.65 wt. % essence such as linalool, linalyl acetate, or as combination thereof. The composition may be aerosilized in combination with up to at least one of 15 wt. % propane or 35 wt. % butane.

The present disclosure provides a composition which may comprise, in a mixture, 35-45 wt. % ethyl alcohol, 4-9 wt. % 1,2-propanediol, and essence such as 0.1-0.5 wt. % linalool, 0.1-0.5 wt. % linalyl acetate, or as a combination thereof.

The present disclosure provides a method of treating an environment by providing a composition which may comprise, in a mixture, 35-45 wt. % ethyl alcohol, 4-9 wt. % 1,2-propanediol, and essence. The composition may be aerosilized in combination with 0.6-1.8 wt. % dipropylene glycol, 3-17 wt. % propane, 31-39 wt. % butane and applied to an environment in an effective amount to treat the environment such that airborne bacteria and odor are illuminated.

The present disclosure provides a method of making a composition which may comprise, in a mixture, 35-45 wt. % ethyl alcohol, 4-9 wt. % 1,2-propanediol, 0.6-1.8 wt. % dipropylene glycol, 13-17 wt. % propane, and 31-39 wt. % butane.

The compositions disclosed may be aerosolized according to conventional means via the inclusion of propellants such as propane, butane, or any other suitable propellant. The compositions disclosed may be aerosolized and stored in a suitable can or bottle under pressure. The can or bottle may be opened to force the composition and propellant out as the pressurized propellant expands.

The compositions of the present disclosure may be prepared in a vessel, such as but not limited to, a batching cylinder or centrifuge, by preparing an admixture of ethyl alcohol and propylene glycol at a stirring speed ranging from about 100 revolutions per minute to about 200 revolutions per minute. The admixture may be mixed for about 5 minutes. Essence may be added to the admixture and the composition may be mixed for an additional period of time ranging from about 10 minutes to about 20 minutes. The admixture may be provided in an aerosolized spray via the introduction of butane or propane to a pressurized container such that it may be dispersed in air to sanitize and reduce odor.

In some embodiments, the pressurized container is constructed of aluminum to regulate and maintain a suitable temperature of the composition within the container. This aids in preventing the flammable contents from combusting.

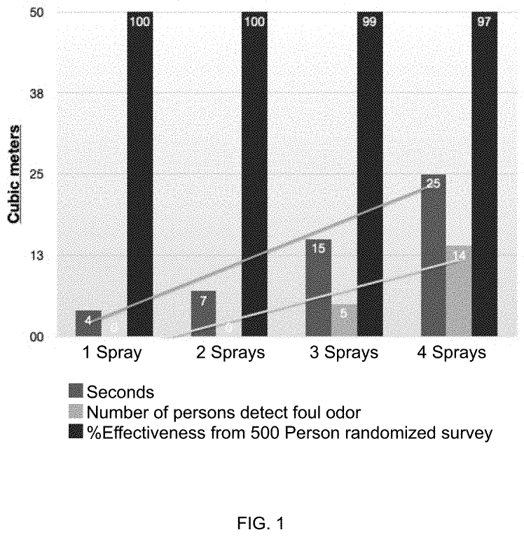

Referring to FIG. 1, the effectiveness of the composition is shown as determined by surveying 500 persons with respect to their ability to detect foul odors after the composition had been used to sanitized and deodorize up to 50 cubic meters of air. The aerosolized composition was sprayed in an approximately 50 cubic meter volume of air and 500 persons were asked to indicate whether the smell of cigarette smoke was detectable. The test was repeated three on three additional occasions increased the amount of composition used to sanitize and deodorize each test. Individual ability to detect foul odors was recorded four seconds, seven second, fifteen seconds, and twenty-five seconds after implementation of the sanitizing and deodorizing composition. Efficacy over time indicates the compositions disclosed herein ranged from approximately 97-100% effectiveness in reducing or eliminating foul odors in the air.

According to variation 1, an admixture may include ethyl alcohol; propylene glycol; and essence.

Variation 2 may include the admixture as described in variation 1 wherein the admixture may include 82.9 wt. % ethyl alcohol, 9.8 wt. % propylene glycol, and 7.3 wt. % essence.

Variation 3 may include the admixture as described in any of variations 1 through 2 wherein the essence may include at least one of linalool or linalyl acetate.

Variation 4 may include the admixture as described in any of variations 1 through 3 wherein the essence may include at least one of 0.1-0.5 wt. % linalool or 0.1-0.5 wt. % linalyl acetate.

Variation 5 may include the admixture as described in any of variations 1 through 4 wherein the admixture may be aerosolized.

Variation 6 may include the admixture as described in any of variations 1 through 5 wherein the admixture may be aerosolized and further may include at least one of propane or butane.

According to variation 7, an aerosolized product may include ethyl alcohol; 1,2-propanediol; an essence comprising at least one of linalool or linalyl acetate; propane; and butane.

Variation 8 may include the aerosolized product as described in variation 7 wherein the aerosolized product may include 35-45 wt. % ethyl alcohol and 4-9 wt. % 1,2-propanediol.

Variation 9 may include the aerosolized product as described in any of variations 7 through 8 wherein the essence may include at least one of 0.1-0.5 wt. % linalool or 0.1-0.5 wt. % linalyl acetate.

Variation 10 may include the aerosolized product as described in any of variations 7 through 9 and may include 13-17 wt. % propane and 31-39 wt. % butane.

According to variation 11, a method of sanitizing and deodorizing may include the steps of providing an admixture of ethyl alcohol, 1,2-propanediol, and essence; and applying the admixture to an environment in an effective amount to treat the environment such that airborne bacteria and odor are reduced.

Variation 12 may include the method of sanitizing and deodorizing as described in variation 11, wherein the admixture may include 35-45 wt. % ethyl alcohol and 4-9 wt. % 1,2-propanediol.

Variation 13 may include the method of sanitizing and deodorizing as described in any of variations 11 through 12 wherein the admixture may include 82.9 wt. % ethyl alcohol and 9.8 wt. % propylene glycol.

Variation 14 may include the method of sanitizing and deodorizing as described in any of variations 11 through 13 wherein the essence may include at least one of 0.1-0.5 wt. % linalool or 0.1-0.5 wt. % linalyl acetate.

Variation 15 may include the method of sanitizing and deodorizing as described in any of variations 11 through 14 wherein the admixture may be in an aerosolized form.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

I claim:

1. An admixture comprising:
ethyl alcohol;
propylene glycol; and
essence.

2. The admixture as set forth in claim 1 wherein the admixture comprises 82.9 wt. % ethyl alcohol, 9.8 wt. % propylene glycol, and 7.3 wt. % essence.

3. The admixture as set forth in claim 1 wherein the essence comprises at least one of linalool or linalyl acetate.

4. The admixture as set forth in claim 1 wherein the essence comprises at least one of 0.1-0.5 wt. % linalool or 0.1-0.5 wt. % linalyl acetate.

5. The admixture as set forth in claim 1 wherein the admixture is aerosolized.

6. The admixture as set forth in claim 1 wherein the admixture is aerosolized and further comprises at least one of propane or butane.

7. An aerosolized product, comprising:
ethyl alcohol;
1,2-propanediol;
an essence comprising at least one of linalool or linalyl acetate;
propane; and
butane.

8. The aerosolized product as set forth in claim 7 comprising 35-45 wt. % ethyl alcohol and 4-9 wt. % 1,2-propanediol.

9. The aerosolized product as set forth in claim 7 wherein the essence comprises at least one of 0.1-0.5 wt. % linalool or 0.1-0.5 wt. % linalyl acetate.

10. The aerosolized product as set forth in claim 7 comprising 13-17 wt. % propane and 31-39 wt. % butane.

11. A method of sanitizing and deodorizing, comprising:
providing an admixture of ethyl alcohol, 1,2-propanediol, and essence; and
applying the admixture to an environment in an effective amount to treat the environment such that airborne bacteria and odor are reduced.

12. The method of sanitizing and deodorizing as set forth in claim 11, wherein the admixture comprises 35-45 wt. % ethyl alcohol and 4-9 wt. % 1,2-propanediol.

13. The method of sanitizing and deodorizing as set forth in claim 11, wherein the admixture comprises 82.9 wt. % ethyl alcohol and 9.8 wt. % propylene glycol.

14. The method of sanitizing and deodorizing as set forth in claim 11, wherein the essence comprises at least one of 0.1-0.5 wt. % linalool or 0.1-0.5 wt. % linalyl acetate.

15. The method of sanitizing and deodorizing as set forth in claim 11, wherein the admixture is in an aerosolized form.
